SEQUENCE LISTING (21 sequences)

SEQ ID NO: 1 - DNA, 405 bp, synthetic construct
  source 1..405: mol_type = other DNA
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 2 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 3 - DNA, 282 bp, synthetic construct
  source 1..282: mol_type = other DNA
atgatccatctgggtcacatcctcttcctgcttttgctcccagtggctgcagctcagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggctga

SEQ ID NO: 4 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 5 - DNA, 693 bp, synthetic construct
  source 1..693: mol_type = other DNA
cccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 6 - protein, 231 aa, synthetic construct
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 78 bp, synthetic construct
  source 1..78: mol_type = other DNA
ttttgggtgctggtggtggttggtggagtcctggcttgctatagcttgctagtaacagtggcctttattattttctgg

SEQ ID NO: 8 - protein, 26 aa, synthetic construct
FWVLVVVGGVLACYSLLVTVAFIIFW

SEQ ID NO: 9 - DNA, 126 bp, synthetic construct
  source 1..126: mol_type = other DNA
gtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 10 - protein, 42 aa, synthetic construct
VRSKRSRLLHSDYMNMTPRRPGPTRKHYQPYAPPRDFAAYRS

SEQ ID NO: 11 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 12 - protein, 42 aa, synthetic construct
KRGRKKLLYIFKQPFMRPVQTTQEEDGCSCRFPEEEEGGCEL

SEQ ID NO: 13 - DNA, 336 bp, synthetic construct
  source 1..336: mol_type = other DNA
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 14 - protein, 112 aa, synthetic construct
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 238 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGSTSGGGSGGGSGGGGSSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIK

SEQ ID NO: 16 - protein, 20 aa, synthetic construct
MYRMQLLSCIALSLALVTNS

SEQ ID NO: 17 - DNA, 51 bp, synthetic construct
  source 1..51: mol_type = other DNA
atgccgctgctgctactgctgcccctgctgtgggcaggggccctggctatg

SEQ ID NO: 18 - protein, 17 aa, synthetic construct
MPLLLLLPLLWAGALAM

SEQ ID NO: 19 - DNA, 578 bp, synthetic construct
  source 1..578: mol_type = other DNA
ctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacat

SEQ ID NO: 20 - DNA, 588 bp, synthetic construct
  source 1..588: mol_type = other DNA
gtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 63 bp, synthetic construct
  source 1..63: mol_type = other DNA
ggctctggggagggcagaggcagcctgctgacctgtggggatgtggaggagaaccctggcccc

(19) United States
(12) Patent Application Publication
(10) Pub. No.: US 2026/0248853 A1
(43) Pub. Date: Aug. 27, 2026

(54) METHOD AND DRUG FOR TREATING CANCER BY JOINT USE OF NKG2D CHIMERIC ANTIGEN RECEPTOR AND PD1 INHIBITOR

(71) Applicant: DAREN BIOTECH LIMITED, Hongkong Hong Kong (CN)

(72) Inventor: Yong XIE, Hongkong Hong Kong (CN)

(21) Appl. No.: 18/994,260

(22) PCT Filed: Jul. 14, 2023
(86) PCT No.: PCT/CN2023/107566

§ 371 Date: Jan. 14, 2025

(30) Foreign Application Priority Data
Jul. 14, 2022 (CN) 202210823835.1

(51) Int. Cl. A61K 35/17; A61K 40/11; A61K 40/31; A61K 40/42; A61P 35/00; C07K 14/705; C07K 16/28; C12N 15/85

(52) U.S. Cl. CPC A61K 35/17; A61K 40/11; A61K 40/31; A61K 40/421; A61P 35/00; C07K 14/7051; C07K 14/70517; C07K 14/70521; C07K 14/70578; C07K 16/2818; C07K 16/2851; C12N 15/85; A61K 2239/13; A61K 2239/21; A61K 2239/22; A61K 2239/29; A61K 2239/48; A61K 2239/55; C07K 2317/31; C07K 2317/52; C07K 2319/036

(57) ABSTRACT

The present disclosure provides a novel chimeric antigen receptor and a combination thereof with a PD1 inhibitor. Further provided are expression vectors and host cells expressing the chimeric antigen receptor and the composition of said receptor with the PD1 inhibitor. Further provided are uses of the chimeric antigen receptor and the composition of said receptor with the PD1 inhibitor in treating cancer or preparing a drug for treating cancer. The drug and the method provided by the present invention can effectively treat cancer, particularly myeloma and solid tumors.

### REFERENCE TO SEQUENCE LISTING SUBMITTED ON COMPUTER

The content of the ASCII text file of the sequence listing named “LHC2560018PUS-250113-SequenceListing” which was filed in PCT/CN2023/107566 on Jul. 14, 2023, downloaded from the WIPO database, is 23 kb in size with a created date of Jan. 14, 2025, and electronically submitted via Patent Center herewith the application, is incorporated herein by reference in its entirety.

The present application claims priority to Chinese Patent Application No. 202210823835.1, with a translated title “Method and drug for treating cancer by joint use of Chimeric antigen receptor and PD1 inhibitor” filed on Jul. 4, 2022, the disclosure of which is hereby incorporated into the present application by reference in its entirety.

### FIELD OF THE INVENTIONS

The present disclosure relates to the field of immunology and pharmacy field. In particular, the disclosure provides a new chimeric antigen receptor and a combination thereof with PD1 inhibitor. The disclosure also provides a method of using said combination of chimeric antigen receptor and PD1 inhibitor for treating cancer, and pharmaceutical compositions for treating cancer and a manufacturing method thereof.

### BACKGROUND

Adoptive cell transfer (ACT), as a method of immunotherapy, has shown remarkable results in the treatment of hematological malignancies and malignant melanoma. Chimeric Antigen Receptor T Cells (CAR T) use genetically modified T cells to express chimeric antigen receptors (CAR) that specifically target tumor-associated antigens (TAAs), shown in clinical trials to treat B-cell malignancies produced encouraging results.

NKG2D (natural-killer group 2 member D, or NKG2D receptor), killer cell lectin-like receptor subfamily K member 1, is type II expressed on all natural killer cells, natural killer T cells and γδ⁺ T cells Transmembrane protein. In humans, NKG2D receptors mainly bind to two ligands, namely UL16-binding protein (ULBP) and MHC class I-chain associated protein A/B (MHC class I-chain-related protein, MICA/B). In natural killer cells and T cells, DNAX activating protein 10 (DAP10) is the cell surface adapter of the NKG2D receptor. There have been applications in the art of NKG2D receptor-NKG2D ligand systems for chimeric antigen receptor (CAR) therapy. WO2019/192526A1 discloses the use of a combination of NKG2D chimeric antigen receptor and DAP10 in a method for treating cancer, in which the NKG2D chimeric antigen receptor includes the aa. 82-216 fragment of NKG2D; the IgG1 heavy chain constant as the hinge region; CD8 transmembrane domain; CD28 The intracellular signaling domain of 4-1BB and the intracellular signaling domain of CD3ζ.

There is a negative impact of the immunosuppressive microenvironment (TME) on CAR T cells in cancer, especially in solid tumors. This environment increases inhibitory receptors (IR) on T cells, such as: cytotoxic T lymphocyte-associated protein 4 (CTLA-4), T cell immunoglobulin domain and mucin domain-containing protein 3 (TIM-3; also known as HAVCR2), lymphocyte activating gene 3 (LAG-3), and programmed death 1 (PD-1). These molecules cause T cell dysfunction and exhaustion after sustained activation, thereby leading to tumor escape. PD-1 (programmed death 1, programmed death molecule-1) on the surface of immune cells interacts with the immunoglobulin-like molecule PD-L1 (programmed cell death-Ligand 1, programmed cell death-ligand 1) produced on the surface of tumor cells.) or PDL-2 combine with each other, and the combination produces molecular signals that reduce the activity of immune cells (such as T cells), thus blocking the attack of immune cells on tumor cells. Tumors use this method to hide themselves and therefore survive.

Therefore, more effective CAR-T cells and improved adoptive cell transfer therapies are needed in the field against different types of tumors.

### SUMMARY OF THE INVENTIONS

The present disclosure finds that CAR-T cells with NKG2D antigen receptor structure and/or its accessory protein DAP10 can effectively recognize cancer cells with NKG2D ligands and activate tumor cell-specific anti-tumor cellular immune responses and killing tumor cells. The present disclosure also proves for the first time that the CAR-T cells provided by the present disclosure with a NKG2D antigen receptor and/or its accessory protein DAP10 in combination with an antibody that blocks the binding of PD-1 to PD-L1 or PD-L2 or Its active fragment can significantly enhance the effect of killing tumors, especially solid tumors, including lung cancer, liver cancer, myeloma, etc.

Specifically, the invention provides an immune cell expressing: i) a chimeric antigen receptor (CAR), and ii) an antibody or active fragment thereof that blocks the binding of PD-1 to PD-L1 or PD-L2. The chimeric antigen receptor includes: (a) an antigen-binding domain, which includes NKG2D or an active fragment thereof; (b) a transmembrane domain and (c) an intracellular signaling domain. The chimeric antigen receptor described in the present invention is used in combination with its accessory protein DAP10. The present invention also provides nucleic acids and expression vectors encoding and expressing the chimeric antigen receptor and/or DAP10 and the antibody or active fragment thereof that blocks the binding of PD-1 to PD-L1 or PD-L2. The present invention also provides methods for treating cancer or for preparation of medicaments for treating cancer by using the immune cells, the nucleic acids or vectors encoding the antibodies or active fragments thereof that express the chimeric antigen receptor and/or DAP10 and block the binding of PD-1 to PD-L1 or PD-L2.

In one aspect of the invention, a new immune cell is provided expressing:
i) Chimeric Antigen Receptor (CAR) and its accessory proteins, and
ii) Antibodies or active fragments thereof that block the binding of PD-1 to PD-L1 or PD-L2.

In one aspect of the invention, the CAR comprises: (a) an antigen-binding domain, which includes NKG2D, preferably a.a.82-216 fragment of NKG2D, or an active fragment thereof; (b) a transmembrane domain and (c) an intracellular signaling domain. In one aspect of the invention, the accessory protein of the CAR is DAP10.

In one aspect of the present invention, the chimeric antigen receptor (CAR) and its accessory protein are used in combination, for example, the engineered immune cells express the CAR and the accessory protein simultaneously. Wherein, the accessory protein is DAP10 or its active fragment. In one aspect of the invention, the DAP10 has the amino acid sequence of SEQ ID NO: 4.

As used herein, the term “NKG2D”, also known as “NKG2-D”, “CD314”, “KLRK1”, “killer-cell lectin-like receptor subfamily K, member 1”, refers to the killer cell lectin-like receptor subfamily K, member 1 (KLRK1, such as NCBI RefSeq NM_007360) of mammals, especially humans or gene products thereof (such as NCBI RefSeq NP_031386) or naturally occurring variants thereof.

DAP10 (membrane protein 10) is a cell surface adaptor protein, which can form an activating receptor complex with NKG2D, particular refer to humans DAP10 (GenBank: AAG29425.1). The activity of DAP10 includes forming a complex with NKG2D (Wu, J. et al., Science 285 (5428), 730-732, 1999).

In certain embodiments of the present disclosure, the antigen binding domain of said chimeric antigen receptor (CAR) comprises a functional fragment of NKG2D. Said functional fragment can be, for example, an NKG2D fragment corresponding to amino acid residues 82-216 of human NKG2D, that is, an NKG2D fragment comprising amino acids No. 82-216 in the amino acid sequence as set forth in SEQ ID NO: 2:

LFNQEVQIPLTESYCGPCPKNWICYKNNCYQFFDESKNWYESQASCMSQNASLLKVYSKEDQ
DLLKLVKSYHWMGLVHIPTNGSWQWEDGSILSPNLLTIIEMQKGDCALYASSFKGYIENCSTP
NTYICMQRTV.

In certain embodiments of the present disclosure, the antigen binding domain of said chimeric antigen receptor (CAR) comprises a leader peptide. A leader peptide assists the expression of a protein and transports the protein through the cell membrane. Leading peptides known in the field can be used in the present disclosure. In the antigen binding domain of CAR of the present disclosure, the leading peptide can be located upstream of NKD2G or its functional fragments. In certain embodiments of the present disclosure, said leader peptide is a leader peptide of CD33, which has an amino acid sequence of SEQ ID NO: 18. A leader peptide can assist the expression of a CAR on the surface of a cell, yet the leading peptide is not necessary for the CAR to function. In the embodiments of the present disclosure, after expression of CAR on the surface of cells, the leader peptide is cleaved from CAR. In certain embodiments of the present disclosure, said CAR does not include a leader peptide.

In certain embodiments of the present disclosure, said chimeric antigen receptor (CAR) comprises a transmembrane domain. In yet one aspect of the present disclosure, transmembrane domains can be used in the present disclosure include transmembrane domain of T cell receptor α, β, or ζ, CD28, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1(CD11a, CD18), ICOS(CD278), 4-1BB(CD137), GITR, CD40, BAFFR, HVEM(LIGHTR), SLAMF7, NKp80(KLRF1), or the like.

In certain embodiments of the present disclosure, said transmembrane domain of the CAR comprises a transmembrane domain of i) CD8 and/or ii) CD28. In yet some other embodiments of the present disclosure, said transmembrane domain of the CAR comprises a transmembrane domain of CD8. In yet some other embodiments of the present disclosure, said transmembrane domain of the CAR is a transmembrane domain of CD8.

Preferably, a transmembrane domain used in the chimeric antigen receptor (CAR) of the disclosure can include one or more of a intracellular signaling of i) CD28, ii) 4-1BB, and/or iii) CD3ζ. In preferred embodiments of the present disclosure, the intracellular signaling used in the CAR of the disclosure includes CD28, 4-1BB, and CD3ζ. In more preferred embodiments of the present disclosure, the intracellular signaling used in the CAR of the disclosure comprises in the order from the N terminal to the C terminal: CD28, 4-1BB, and CD3ζ. Herein, CD28 is a T cell marker important in T cell co-stimulation. 4-1BB, also known as CD 137, transmits a potent costimulatory signal to T cells, promoting differentiation and enhancing long-term survival of T lymphocytes. CD3ζ associates with TCRs to produce a signal and contains immunoreceptor tyrosine-based activation motifs (ITAMs). In certain embodiments of the present disclosure for the intracellular signaling domain, CD28 in the CAR has an amino acid sequence of SEQ ID NO: 10. In one aspect of the present disclosure, the intracellular signaling domain of 4-1BB in the CAR has an amino acid sequence of SEQ ID NO: 12. In certain embodiment of the present disclosure, the intracellular signaling domain of CD3 in the CAR has an amino acid sequence of SEQ ID NO: 14.

In a CAR comprising several intracellular signaling domains, an oligo linker or a peptide linker can be placed between each two intracellular signaling domains. Preferably, the linker is a peptide comprising 2-10 amino acids. For example, the linker is a peptide comprising glycine-serine.

In one aspect of the present disclosure, the chimeric antigen receptor (CAR) of the disclosure comprises: (a) an antigen binding domain including an NKG2D fragment corresponds to amino acid residues 82-216 of human NKG2D; (b) a transmembrane domain of CD28; and (c) an intracellular signaling domain which comprises, in the order from the N terminal to the C terminal, CD28, 4-1BB, and CD3ζ.

In certain embodiments of the present disclosure, said chimeric antigen receptor (CAR) includes a hinge region between said (a) antigen binding domain and said (b) transmembrane domain. Hinge regions known in the art may be used in the present invention, including CD8a hinges, IgG1 hinges, or FcγRII hinges, and the like. Hinge regions can be used in the CAR of the present disclosure includes IgG1H, IgG2H, IgG3H or IgG4H, and the like. In one aspect of the invention, the hinge region is an IgG heavy chain constant region sequence (IgGHc) such as IgG1Hc, IgG2Hc, IgG3Hc, IgG4Hc, etc. In yet one aspect of the present disclosure, the hinge region between said (a) antigen binding domain and said (b) transmembrane domain comprises IgG1Hc or a fragment thereof or a variant thereof. For example, the hinge region has an amino acid sequence of SEQ ID NO: 6.

Functional variants of the CARs or the functional fractions thereof (including the antigen binding domain, transmembrane domain, intracellular signaling domain, hinge region and leading peptide) are included in the scope of the disclosure. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, about 50%, about 75%, about 80%, about 90%, about 98%), about 99% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

In one aspect of the invention, the engineered immune cells provided by the invention also express ii) an antibody or active fragment thereof that blocks the binding of PD-1 to PD-L1 or PD-L2.

“PD-1” is programmed cell death protein 1, also known as CD279, which is the cell surface receptor for PD-L1. PD-1 binds two ligands, PD-L1 and PD-L2. PD-1 is a transmembrane protein that contains an extracellular domain, followed by a transmembrane region and an intracellular domain. As used herein, PD-1 may include full-length and/or unprocessed PD-1 as well as any intermediates resulting from processing in the cell, as well as PD-1 variants, e.g., splice variants or alleles Variants. Show The amino acid sequence of an exemplary human PD-1 protein can be found, for example, under NCBI Protein Database Accession No. NP-005009.

“PD-L1” refers to programmed cell death ligand 1, also known as CD274 or B7-H1. Natural PD-L1 includes two extracellular domains, a transmembrane domain and a cytoplasmic domain. The amino acid sequence of an exemplary human full-length PD-L1 protein can be found, for example, under NCBI Protein Data Bank Accession No. NP-054862. “PD-L2” refers to programmed cell death 1 ligand 2, also known as CD273. The amino acid sequence of an exemplary human full-length PD-L2 protein can be found, for example, under NCBI Protein Database Accession No. NP-079515.

PD-1 is a negative immunomodulator that activates T cells when engaged with the ligands PD-L1 and PD-L2. Upregulation of PD-L1 is a mechanism by which tumor cells can evade the host immune system. PD-1 blockade by antagonist antibodies induces anti-tumor responses mediated through the host's endogenous immune system.

Various PD-1/PD-L1 inhibitors or PD-1/PD-L2 inhibitors can be used in the present invention. PD-1/PD-L1 inhibitors refer to agents that disrupt the PD-1/PD-L1 signaling pathway, including biological macromolecules or small chemical molecules. In some embodiments, the inhibitor inhibits the PD-1/PD-L1 signaling pathway by binding to PD-1 and/or PD-L1. In some embodiments, the inhibitor also binds PD-L2. In some embodiments, PD-1/PD-L1 inhibitors block the binding of PD-1 to PD-L1 and/or PD-L2. The inhibitor may be, for example, an antibody, a fusion protein, or a small molecule inhibitor of the PD-1/PD-L1 signaling pathway.

In one aspect of the invention, the ii) antibody or active fragment thereof that blocks the binding of PD-1 to PD-L1 or PD-L2 is a PD-1 antibody or an active fragment thereof, for example, a PD-1 antibody Single chain variable fragment (scFv), Fab, F(ab′)2, Fv, Fd or dAb.

As used herein, “antibody” refers to an immunoglobulin or fragment or derivative thereof, and encompasses any polypeptide comprising an antigen-binding site, whether produced in vitro or in vivo. The term includes, but is not limited to, polyclonal, monoclonal, monospecific, multispecific, nonspecific, humanized, single-stranded, chimeric, synthetic, recombinant, hybrid, Mutated and transplanted antibodies. As used herein, the term “antibody” also includes antibody fragments such as Fab, F(ab′)2, Fv, scFv, Fd, dAb, and others that retain antibody activity (i.e., antigen-binding function such as the ability to specifically bind PD-1).

As used herein, “antibody fragment” refers to (i) monovalent and monospecific antibody derivatives including variable heavy and/or light chains of an antibody or functional fragments and lacking the Fc portion; and (ii) BiTE (tandem scFv), DART, diabodies and single chain diabodies (scDB). Thus, an antibody fragment is, for example, selected from the group consisting of: Fab, Fab′, scFab, scFv, Fv fragment, Nanobody, VHH, dAb, minimal recognition unit, single chain diabody (scDb), BiTE and DART. The antibody fragments have a molecular weight below 60 kDa.

The subunit structure and three-dimensional conformation of different types of antibodies are well known in the art. Briefly, each light chain consists of an N-terminal variable domain (VL) and a constant domain (CL). Each heavy chain consists of an N-terminal variable domain (VH), three or four constant domains (CH), and a hinge region. The CH domain closest to VH is named CH1. The VH and VL domains are composed of four regions with relatively conserved sequences, called framework regions (FR1, FR2, FR3, and FR4), which form a scaffold for three hypervariable sequence regions called complementary decision region (CDR). CDRs contain most of the residues responsible for specific interactions with antigens. The three CDRs are called CDR1, CDR2 and CDR3. The CDR components on the heavy chain are called H1, H2, and H3, while the CDR components on the light chain are correspondingly called L1, L2, and L3. CDR3, and particularly H3, is the greatest source of molecular diversity within antigen-binding domains. For example, H3 can be as short as 2 amino acid residues or more than 26 amino acid residues.

Fab fragments (antigen-binding fragments) consist of VH-CH1 and VL-CL domains covalently linked by disulfide bonds between constant regions. To overcome the tendency for non-covalently linked VH and VL domains in an Fv to dissociate when co-expressed in host cells, single-chain (sc) Fv fragments (scFv) can be constructed. In scFv, a flexible and sufficiently long polypeptide either links the C-terminus of VH to the N-terminus of VL, or the C-terminus of VL to the N-terminus of VH. Most commonly, the 15 residue (Gly4Ser)3 peptide is utilized as the linker, but other linkers are also known in the art.

Typically, such fragments contain an antigen-binding domain. The antigen-binding domain typically includes an antibody light chain variable region (VL) and an antibody heavy chain variable region (VH), although it does not necessarily include both. For example, so-called Fd antibody fragments consist only of the VH domain but still retain some of the antigen-binding functions of the intact antibody.

In some embodiments, an antibody that inhibits PD-1 binds to PD-1 and blocks the binding of PD-L1 and/or PD-L2 to PD-1.

The term “blocks binding of a ligand” is used to refer to the ability to inhibit the interaction between PD-1 and a PD-1 ligand, such as PD-L1. Such inhibition may occur through any mechanism, including direct interference with ligand binding, e.g., because of overlapping binding sites on PD-1, and/or conformational changes in PD-1 induced by the antibody that alter ligand affinity, etc. Antibodies and antibody fragments referred to as “functional” are characterized by having such properties.

In some embodiments, an antibody that inhibits PD-1 is an antibody that recognizes and binds PD-1, which inhibits PD-1 activity. PD-1 activity refers to one or more immunomodulatory activities related to PD-1. PD-1 is a negative regulator of TcR/CD28-mediated immune responses.

Antibodies that inhibit PD-1 or anti-PD-1 antibodies can be prepared or identified by various methods known in the art. In general, for example, traditional hybridoma technology, recombinant DNA methods, or phage libraries can be used for preparation of antibodies.

Various PD-1-inhibiting antibodies disclosed in the art, such as Nivolumab, Pembrolizumab, or PDR001, can be used in the present invention. Exemplary PD-1 antibodies useful in the present invention are Nivolumab, and active fragments of Nivolumab, such as its single chain variable fragment (scFv), Fab, F(ab′)2, Fv, Fd or dAb, these fragments can include the heavy and light chains, or various combinations of CDR1, CDR2 and CDR3 of the heavy and light chains of Nivolumab, and have the activity of recognizing and binding to PD-1. Nivolumab is described in U.S. Pat. No. 8,008,449 and Wang et al., 2014 Cancer Immunol Res. 2(9):846-56. Nivolumab is a fully human IgG4 (S241P) anti-PD-1 antibody that selectively blocks the interaction of PD-1 with its ligands PD-L1 and PD-L2, thereby inhibits the downregulation of anti-tumor T cell function.

In some embodiments, an exemplary anti-PD-1 antibody for use in the present invention is a single chain variable fragment (scFv) of nivolumab, which includes the heavy and light chains of nivolumab. In some embodiments, the scFv of an exemplary nivolumab used in the present invention has the amino acid sequence of SEQ ID NO: 15. The heavy chain and light chain are connected through the GS linking sequence (GSTSGGGSGGGSGGGGSS).

In yet another aspect of the present invention, said ii) antibody or active fragment thereof that blocks the binding of PD-1 to PD-L1 or PD-L2 is secreted. For example, the PD-1 antibody or active fragment thereof has a signal peptide. Signal peptides are 5 to 30 amino acid peptides attached to the N-terminus of proteins to be secreted and are attached to increase protein secretion. In a preferred embodiment, the signal peptide is an IL-2 signal peptide. In some embodiments, the signal peptide has the amino acid sequence of SEQ ID NO: 16.

The present invention also provides nucleic acids encoding the above-mentioned chimeric antigen receptor (CAR) and/or its accessory proteins such as DAP10, as well as antibodies or active fragments thereof that block the binding of PD-1 to PD-L1 or PD-L2, and vectors comprising such nucleic acids.

The nucleic acid provided by the present invention may include a nucleotide sequence encoding the leader sequence, antigen-binding domain, transmembrane domain and/or intracellular T cell signaling domain, hinge region, etc. of the chimeric antigen receptor (CAR) described above; a nucleotide sequence encoding an auxiliary protein of the chimeric antigen receptor (CAR) described above, such as DAP10 or an active fragment thereof; and a nucleotide sequence encoding an antibody or its active fragment as previously described blocking the binding of PD-1 to PD-L1 or PD-L2, such as the nucleotide sequence of its heavy chain or light chain, its heavy chain variable region or light chain variable region; or any combination thereof.

In one aspect of the present disclosure, the present disclosure provides an isolated nucleic acid comprising a nucleotide sequence encoding any of the CARs described above, which comprises: (a) an antigen binding domain including NKG2D or a functional fragment thereof; (b) a transmembrane domain, and (c) an intracellular signaling domain.

In certain embodiments of the present disclosure, the nucleotide sequence encoding antigen binding domain comprises a nucleic acid sequence encoding an NKG2D functional fragment, preferably an NKG2D fragment corresponds to amino acid residues 82-216 of human NKG2D, for example, the nucleic acid sequence encoding an NKG2D functional fragment comprises a sequence of SEQ ID NO: 1.

In certain embodiments of the present disclosure, the nucleotide sequence encoding the transmembrane domain comprises the nucleotide sequence encoding a transmembrane domain of CD8 and/or CD28. In yet some other embodiments of the present disclosure, the nucleotide sequence encoding the transmembrane domain comprises the nucleotide sequence encoding a transmembrane domain of CD28, for example, the nucleotide sequence comprises a sequence of SEQ ID NO: 7.

In certain embodiments of the present disclosure, the nucleotide sequence encoding the intracellular signaling domain comprises one or more of the nucleotide sequence encoding an intracellular signaling domain of CD28, 4-1BB or CD3ζ. In yet some other embodiments of the present disclosure, the nucleotide sequence encoding the intracellular signaling domain comprises the nucleotide sequence encoding an intracellular signaling domain including CD28, 4-1BB and CD3ζ, more preferably, the intracellular signaling domain contains or consists of intracellular signaling domains of CD28, 4-1BB and CD3ζ from the N terminal to the C terminal.

In certain embodiments of the present disclosure, the nucleotide sequence encoding the intracellular signaling domain of CD28 in the CAR comprises a sequence of SEQ ID NO: 9.

In certain embodiments of the present disclosure, the nucleotide sequence encoding the intracellular signaling domain of 4-1BB in the CAR comprises a sequence of SEQ ID NO: 11.

In certain embodiments of the present disclosure, the nucleotide sequence encoding the intracellular signaling domain of CD3ζ in the CAR comprises a sequence of SEQ ID NO: 13.

In certain embodiments of the present disclosure, the nucleotide sequence encoding the intracellular signaling domain in the CAR comprises a nucleotide sequence encoding intracellular signaling domains of CD28, 4-1BB and CD3ζ from the N terminal to the C terminal. For example, said nucleotide sequence encoding the intracellular signaling domain in the CAR comprises a sequence of SEQ ID NO: 20.

In certain embodiments of the present disclosure, said isolated nucleic acid comprises a nucleotide sequence encoding a hinge region between said antigen binding domain and said transmembrane domain, and preferably the hinge region is an IgG1Hc. For example, the nucleotide sequence encoding a hinge region comprises a sequence of SEQ ID NO: 5.

In certain embodiments of the present disclosure, said isolated nucleic acid comprises a nucleotide sequence encoding a leader sequence upstream said NKG2D fragment, and preferably, the leader sequence is a leading peptide of CD33. For example, the nucleotide sequence encoding a leader sequence comprises a sequence of SEQ ID NO: 17.

In one aspect, the present disclosure provides an isolated nucleic acid encoding the chimeric antigen receptor (CAR) and the accessory protein, wherein said CAR comprises: (a) an antigen binding domain including NKG2D or a functional fragment thereof; (b) a transmembrane domain; and (c) an intracellular signaling domain. Herein said accessory protein can be DAP10 or a functional fragment thereof. In certain embodiments of the present disclosure, the nucleotide sequence encoding antigen binding domain comprises a nucleic acid sequence encoding an NKG2D functional fragment, preferably an NKG2D fragment corresponding to amino acid residues 82-216 of human NKG2D. For example, the nucleic acid sequence encoding an NKG2D functional fragment comprises a sequence of SEQ ID NO: 1, and the nucleotide sequence encoding DAP10 comprises a sequence of SEQ ID NO: 3.

In yet another aspect of the present invention, the nucleic acid provided by the present invention also includes a nucleotide sequence encoding an antibody or active fragment thereof that blocks the binding of PD-1 to PD-L1 or PD-L2. In yet another aspect of the invention, the nucleic acid includes a nucleotide sequence encoding a single chain variable fragment (scFv), Fab, F(ab′)2, Fv, Fd or dAb of the antibody. In yet another aspect of the invention, the nucleic acid includes a nucleotide sequence encoding the heavy chain and/or light chain of the antibody. In yet another aspect of the present invention, the nucleic acid includes a nucleotide sequence encoding the heavy chain variable region and/or the light chain variable region of the antibody, or its CDR, or any combination thereof.

In yet another aspect of the present invention, the nucleic acid provided by the present invention also includes at least one IRES sequence and/or at least one 2A sequence, such as P2A, T2A, E2A and F2A. It helps separate multiple ORFs on a nucleic acid fragment, so that a single mRNA transcript will produce multiple proteins. In one embodiment of the present invention, there is a 2A sequence, such as a nucleotide sequence of T2A, between the nucleic acid fragment encoding the CAR and/or the DAP10 and the antibody or active fragment thereof that blocks the binding of PD-1 to PD-L1 or PD-L2. In one embodiment of the present invention, there is a 2A sequence, such as a nucleotide sequence of P2A, between the nucleic acid fragment encoding the CAR and the DAP10. In one embodiment of the present invention, there is a nucleotide sequence of IRES between the nucleic acid fragment encoding the CAR and the DAP10.

The present disclosure further provides isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The disclosure also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

The nucleic acids of the disclosure can be incorporated into a recombinant expression vector. In this regard, certain embodiments of the disclosure provide a recombinant expression vector comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell.

In certain embodiment, the recombinant expression vector of the disclosure can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBIi21 and pBIN19 (Clontech). Examples of animal expression vectors include pEUK-Cl, pMAM, and pMAMneo (Clontech). The recombinant expression vector may be a viral vector, e.g., a retroviral vector or a lenti viral vector.

The recombinant expression vector may include a natural or non-natural promoter, which is operably linked to the nucleotide sequence encoding the CAR (including its functional parts and functional variants) or to a nucleotide sequence that is complementary or hybridized to the nucleotide sequence encoding the CAR nucleotide sequence. The promoter can be a non-viral promoter or a viral promoter, such as EF1α promoter, cytomegalovirus (CMV) promoter, SV40 promoter, and RSV promoter. The EF1α promoter is derived from the EF1α promoter from the human targeted elongation factor 1α (EF1A) gene. In certain embodiments of the present disclosure, the EF1α promoter is used in the recombinant expression vector.

In one aspect of the present disclosure, the present disclosure provides a recombinant expression vector of the aforementioned chimeric antigen receptor (CAR) and accessary protein, as well as antibodies or active fragments thereof that block the binding of PD-1 to PD-L1 or PD-L2. In one aspect of the present disclosure, the present disclosure provides a recombinant expression vector comprising a nucleotide sequence encoding aforementioned chimeric antigen receptor (CAR) and accessary protein, as well as antibodies or active fragments thereof that block the binding of PD-1 to PD-L1 or PD-L2. The CAR comprises: (a) an antigen binding domain including NKG2D or a functional fragment thereof; (b) a transmembrane domain; and (c) an intracellular signaling domain.

In one aspect of the present disclosure, the recombinant expression vector of a chimeric antigen receptor (CAR) and an accessory protein expresses said CAR or said accessary protein in different recombinant expression vectors. In one preferrable embodiment of the present disclosure, the recombinant expression vector of a chimeric antigen receptor (CAR) and an accessory protein expresses said CAR or said accessary protein in one recombinant expression vector.

In one aspect of the present invention, the expression vector of the present invention also includes a transcript upstream of the sequence encoding the antigenic domain.

In one aspect of the present invention, the expression vector of the present invention has a promoter upstream of the sequence encoding the antigenic domain, such as the EF1α promoter.

In one aspect of the invention, in the expression vector of the invention, the nucleic acid encoding the antibody or active fragment thereof that blocks the binding of PD-1 to PD-L1 or PD-L2 includes nucleotide sequence encoding a PD-1 antibody or active fragment thereof, for example, it is the nucleotide sequence of a single-chain variable fragment (scFv), Fab, F(ab′)2, Fv, Fd or dAb of a PD-1 antibody.

In one aspect of the invention, the expression vector of the invention also includes a nucleotide sequence encoding a signal peptide located upstream of the PD-1 antibody or active fragment thereof, preferably a nucleoside encoding the leader sequence of IL-2 acid sequence.

In one aspect of the invention, in the expression vector of the invention, there is a nucleotide sequence of IRES or 2A sequence between the nucleic acid fragment encoding CAR and/or DAP10 and the nucleic acid fragment encoding the antibody or active fragment thereof that blocks the binding of PD-1 to PD-L1 or PD-L2. For example, when the expression vector has sequences encoding CAR and DAP10, there is a nucleotide sequence of IRES or 2A sequence between the nucleic acid fragment encoding DAP10 and the nucleic acid fragment encoding the antibody or active fragment thereof that blocks the binding of PD-1 to PD-L1 or PD-L2. The 2A sequence is such as T2A. The sequence encoding T2A is, for example, the sequence shown in SEQ ID NO: 21.

The present disclosure further provides host cells expressing said chimeric antigen receptor (CAR). The present disclosure provides host cells expressing a combination of said chimeric antigen receptor (CAR) and an accessary protein such as DAP10 and the antibody or active fragment thereof that blocks the binding of PD-1 to PD-L1 or PD-L2. The present disclosure further provides host cells containing the nucleic acid and the recombinant expression vector as mentioned above.

As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC).

In one aspect of the invention, the host cells are immune cells, particularly engineered immune cells. Engineered immune cells refer to immune cells that have been genetically modified to express the proteins described herein. In one aspect of the invention, the cells are autologous. In another aspect of the invention, the cells are allogeneic.

The immune cells may be T cells, natural killer T (NKT) cells, natural killer (NK) cells, human embryonic stem cells, hematopoietic stem cells (HSC), or induced pluripotent stem cells (iPS).

In certain embodiments of the present disclosure, the host cell is a T cell. For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naive T cells, and the like. The T cell may be a CD8⁺ T cell or a CD4⁺ T cell.

In certain embodiments of the present disclosure, the host cell is a natural killer (NK) cell. The term “NK cells” (also known as natural killer cells) refers to a type of lymphocytes that originate in the bone marrow and play an important role in the innate immune system. NK cells provide a rapid immune response against virus-infected cells, tumor cells or other stressed cells, even if antibodies and major histocompatibility complexes are not present on the cell surface. NK cells can be isolated or can be obtained from commercially available sources.

As used herein, “signal sequence” or “leader sequence” refers to the peptide sequence (5, 10, 15, 20, 25, 30 amino acids in length) at the N-terminus of a newly synthesized protein that guides its entry into the secretory pathway.

The CAR substances of the invention can be formulated into pharmaceutical compositions. In this regard, the present disclosure further provides a pharmaceutical composition containing said CAR materials. An embodiment of the disclosure provides a pharmaceutical composition comprising any of the CARs, functional portions, functional variants, nucleic acids, expression vectors, host cells (including populations thereof), and antibodies (including antigen binding portions thereof), and a pharmaceutically acceptable carrier. The inventive pharmaceutical compositions containing any of the inventive CAR materials can comprise more than one inventive CAR material, e.g., a CAR and a nucleic acid, or two or more different CARs. Alternatively, the pharmaceutical composition can comprise an inventive CAR material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In a preferred embodiment, the pharmaceutical composition comprises the inventive host cell or populations thereof.

With respect to pharmaceutical compositions, the pharmaceutically acceptable carrier can be any of those conventionally used and is limited only by chemical-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

For purposes of the inventive methods wherein host cells or populations of cells are administered, the cells can be cells that are allogeneic or autologous to the mammal. Preferably, the cells are autologous to the mammal.

The mammal as referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including bovines (cows) and swines (pigs) or of the order Perssodactyla, including Equines (horses). The mammals may be of the order Primates, Ceboids, or Simoids (monkeys) or of the order Anthropoids (humans and apes). Preferably, the mammal is a human.

In one aspect of the present disclosure, the pharmaceutical composition can be for use in the treatment or prevention of cancer.

The present disclosure also provides the use of the CAR, the combination of the CAR with an accessary protein and an antibody or active fragment thereof that blocks the binding of PD-1 to PD-L1 or PD-L2, or the nucleic acid or the recombinant expression vector or the host cells in treating or preventing cancer, or in manufacturing a medicament for treating or preventing cancer.

The cancer can be any cancer, including leukemia, lymphoma, or solid tumors. For example, leukemia can be acute lymphocytic leukemia, acute myelogenous leukemia, acute promyelocytic leukemia, acute lymphocytic leukemia, chronic myelogenous leukemia, chronic lymphocytic leukemia, monocytic leukemia or hairy cell leukemia. Lymphomas can be Hodgkin's lymphoma, non-Hodgkin's lymphoma; Burkitt's lymphoma; and small lymphocytic lymphoma. Solid tumors can be bladder cancer, urothelial cell carcinoma of urethra, ureter and renal pelvis, myeloma including multiple myeloma, kidney cancer, breast cancer, colon cancer, head and neck cancer, lung cancer, prostate cancer, glioblastoma, osteosarcoma, liposarcoma, soft tissue sarcoma, ovarian cancer, melanoma, liver cancer, esophageal cancer, pancreatic cancer and gastric cancer. In one aspect of the present disclosure, the cancer is NKG2D-related cancer. In said cancer, the NKG2D receptor-NKG2D ligand system is expressed in cancer cells and plays a physiological and biochemical role. In cells of said cancer, NKG2D ligands are usually expressed, including UL16-binding protein (UL16-binding protein, ULBP) or MHC class I-chain-related protein A/B (MHC class I-chain-related protein, MICA/B).

In certain embodiments of the present disclosure, said cancer is liver cancer.

In certain embodiments of the present disclosure, said cancer is lung cancer.

In certain embodiments of the present disclosure, said cancer is myeloma.

### BRIEF DESCRIPTION OF THE DRAWINGS

FIG. 1, including FIGS. 1A to 1C, shows construction diagram of expression vectors. 1A: pcD-NKG2D CAR-DAP10 expression vector; 1B: pcD-NKG2D CAR-DAP10-anti-PD1-A expression vector; 1C: pcD-NKG2D CAR-DAP10-anti-PD1-B expression vector.

FIG. 2 shows the nucleotide sequence and description of the insert fragment of the expression vector pcD-NKG2D CAR-DAP10.

FIG. 3 shows the nucleotide sequence and description of the insert fragment of the expression vector pcD-NKG2D CAR-DAP10-anti-PD1-A.

FIG. 4 shows the nucleotide sequence and description of the insert fragment of the expression vector pcD-NKG2D CAR-DAP10-anti-PD1-B.

FIG. 5, including FIGS. 5A to 5E, shows the effect of CAR-T cells in combination with PD1 antibody active fragments in killing cancer cells. FIG. 5(A)-FIG. 5(E) show the killing effect on various kinds of cancer cells.

FIG. 6, including FIG. 6A to 6C, shows the effect of CART cells and PD1 antibody-binding active fragments co-cultured with target cells to secrete IFN-γ. FIG. 6(A)-FIG. 6(C) shows the effect of co-culture secretion of IFN-γ in various tumor cells.

FIG. 7 is a graph showing BW(g) versus Days post-cell inoculation.

FIG. 8 is a schematic view showing groups versus days post-cell inoculation.

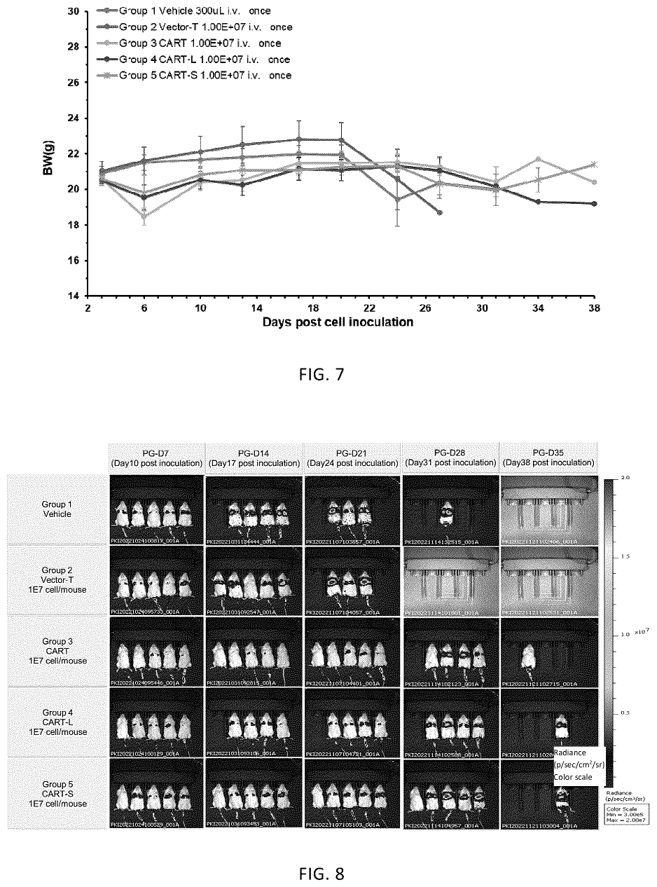

FIG. 9 is a graph showing Survival Percentage versus Days post treatment.

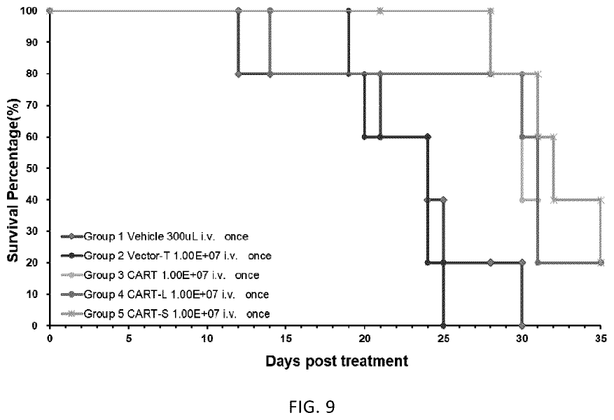

### DETAILED DESCRIPTION OF THE PREFERRED EMBODIMENTS

The inventions provided in the above section of the disclosure are further described with reference to the following examples, which are intended to illustrate the disclosure and not to limit the scope of the disclosure.

### Example 1: Cell Production, Process, and Methods

Buffy coat was obtained from the Hong Kong Red Cross Blood Transfusion Service. Peripheral blood mononuclear cells (PBMCs) were isolated from buffy coat by using Ficoll-Paque PLUS (GE Healthcare). T cells were isolated from PBMCs by using CD3/CD28 Dynabeads (Thermo). T cells isolated from PBMCs were cultured in initiate medium consisting of AIM-V medium (Thermo) supplemented with 5% human serum (Sigma), 2 mM L-glutamine (Thermo) and 50 U/ml IL-2 (Peprotech), or expansion medium consisting of AIM-V medium supplemented with 5% human serum, 2 mM L-glutamine and 300 U/ml IL-2.

All the cell lines mentioned below were obtained from ATCC, ECACC or Chinese Academy of Sciences Cell Bank.

293T cells (ATCC #CRL-3216) were cultured in DMEM medium (Thermo) supplemented with 10% FBS (Thermo), 100 U/ml penicillin (Thermo) and 100 ug/ml streptomycin (Thermo).

NCI-H929 (ATCC #CRL-9608) and U266B1 (ATCC #TIB-196), lung cancer cell line NCI-H522 (ATCC #CRL-5810) were cultured in RPMI1640 medium (Thermo) supplemented with 10% FBS, 100 U/ml penicillin and 100 U/ml streptomycin.

Lung cancer cell line A549 (ATCC #CCL-185) and prostate cancer cell line PC3 (ATCC #CRL-1435) were cultured in F12 medium (Thermo) supplemented with 10% FBS, 100 U/ml penicillin and 100 U/ml streptomycin.

### Construction of Retroviral Plasmids.

### Lentivirus Packaging, Concentration and Purification

Lentivirus was produced by co-transfection of 3rd generation lentiviral plasmid-pMDLg/pRRE, pMD2.G, pRSV-Rev, and transfer plasmids in a ratio of 2:1:1:4 into 293T cells by calcium phosphate transfection method. The freshly collected or thawed supernatant containing lentivirus was put in a centrifuge at 300 g for 3 min to exclude cell debris in the supernatant. The supernatant was filtered by a 0.45-μm minisart syringe filter attached to a 30-ml syringe (TERUMO). The supernatant was centrifuged at 20000 g, 4° C. for 90 minutes. After ultracentrifugation, the supernatant was removed. 1/10 of the initiate lentivirus volume of AIM-V medium was added into centrifuge tubes to re-suspend the pellets. The lentivirus suspension was mixed by pipetting. The concentrated lentivirus was aliquoted and stored in −80° C. freezer.

### Titration of Lentivirus

1×10⁵ Jurkat cells were seeded into each well of 12-well plate in 1 ml RPMI1640 medium supplemented with 10% FBS, 100 U/ml penicillin and 100 ug/ml streptomycin. After overnight culture, different amounts (5 ul to 100 ul) of concentrated lentivirus were added into separate wells. The samples were duplicated or triplicated to increase accuracy. Polybrene (Sigma) was added to a final concentration of 6 ug/ml in each well. 24 hours later, cells were collected by centrifuge and re-suspended in 1 ml fresh RPMI1640 medium supplemented with 10% FBS, 100 U/ml penicillin and 100 ug/ml streptomycin. Another 48 hours later, cells were harvested and the percentage of Jurkat cells expressing CAR was determined by flow cytometry. The titer of lentivirus was calculated by the following formula.

$$Lentivirus Titer ( TU / ml ) = \frac{cell number \times \% of reporter positive cells \times dillution factor}{vector volume ( ml ) \times 100}$$

### T Cells Isolation, Transduction and Cultivation

1×10⁷ PBMC were isolated for CD3+ cells by using Dynabeads coated with antibodies to CD3 and CD28 at a bead-to-cell ratio of 3:1. The cell and bead mixture was incubated at room temperature for 1 hour on a shaker. The CD3+ cells enrichment was performed by magnet and re-suspended in initiation medium at 1×10⁶ cells/ml. 24 hours later, cells were collected by centrifugation (300×g, 3 minutes). The supernatant was discarded. 5×10⁸ TU lentivirus in 500 ul AIM-V medium was added into cells and centrifuged at 2000×g for 2 hours. Cells were re-suspended in the lentivirus culture and 1.5 ml initiation was added. The cells were put back into 6-well plates and placed into an incubator (37 C, 5% CO₂). 24 hours later, Transduction was performed again. Another 24 hours later, cells were collected by centrifuge (300×g, 3 minutes) and re-suspended in 2 ml of expansion medium. The cells were put back into 6-wells plate and placed into incubator (37 degree, 5% CO₂). 72 hours later, cells were transferred to a 100-cm dish and re-suspended in expansion media at concentration of 4×10⁵ cells/ml. Transduction rate can be determined by using Flow cytometer and cytotoxicity assay can be performed when T cells are sufficiently transduced.

### Protein Expression and Flow Cytometry Analysis

To detect CAR expression on the cell surface (both T cells and Jurkat cells), 1×10⁶ cells were re-suspended in 1 ml PBS buffer and stained with biotin goat anti-human IgG(H+L) (Jason Lab) followed by streptavidin-PE (eBioscience).

### Cytotoxicity Assay

Target cells were collected by centrifuge and re-suspended in PBS at a concentration of 1×10⁶ cells/ml. 5 ml of the cells were stained with 2.5 ul of Oregon Green 488 (Thermo) for 20 minutes at 37 C. 20 ml of culture medium was added to absorb the excess dye. Target cells were re-suspended in culture medium at concentration of 4×10⁵ cells/ml.

T cells were collected by centrifuge and re-suspended with the medium required by the target cells at a concentration of 1.6×10⁶ cells/ml.

T cells and target cells were mixed in a ratio of 4, 2, 1 and 0.5 T-cells per target cell.

| E:T ratio | 4:1 | 2:1 | 1:1 | 0.5:1 |
|---|---|---|---|---|
| target cell | 500 μL | 500 μL | 500 μL | 500 μL |
| T cell | 500 μL | 250 μL | 125 μL | 62.5 μL |

Cells were incubated using an 5% CO₂ incubator for 24 hours in 37° C. Cells were collected by centrifuge and re-suspended in 500 ul 7-AAD solution (1 ug/ml). Cells were incubated on ice for 30 minutes. Mortality was analyzed by flow cytometer (7-AAD: excitation wavelength 561 nm, emission wavelength 670 nm).

### Example 2: Construction of Plasmids Expressing NKG2D CAR or Co-Expressing Anti-PD1

1. Construction of pcD-NKG2D CAR-DAP10

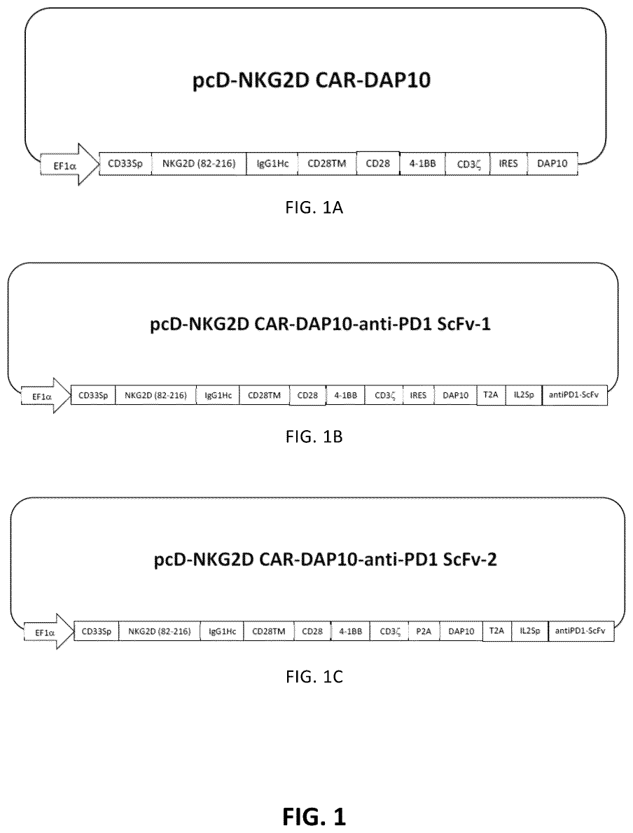

pcD-NKG2D CAR-DAP10 has the structure shown in FIG. 1A.

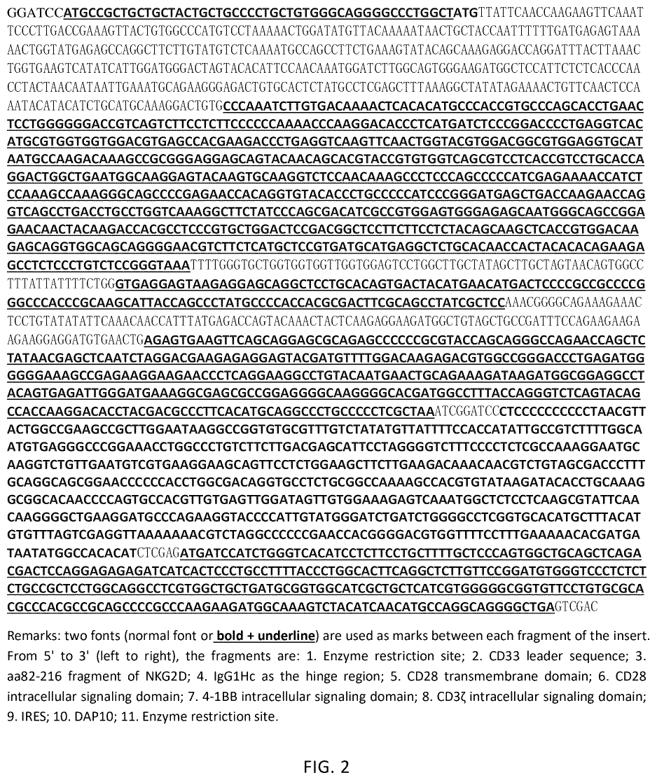

pcD-NKG2D CAR-DAP10 was prepared based on pCDH plasmid, which has a nucleic acid fragment as shown in FIG. 2 encoding NKG2D CAR and DAP10. The nucleic acid fragment includes, from 5′ to 3′, the fragments: 1. CD3ζ leader sequence; 2. aa82-216 fragment of NKG2D; 3. IgG1Hc as the hinge region; 4. CD28 transmembrane domain; 5. CD28 intracellular signaling domain; 6. 4-1BB intracellular signaling domain; 7. CD3ζ intracellular signaling domain; 8. IRES; 9. DAP10.

The above nucleic acid fragment was inserted downstream of the EF1α promoter of pCDH plasmid. The plasmid with the inserted fragment was transformed into competent E. coli, spreaded on a plate, and single clones were picked for sequencing and identification on the next day.

2. Construction of pcD-NKG2D CAR-DAP10-anti-PD1-A

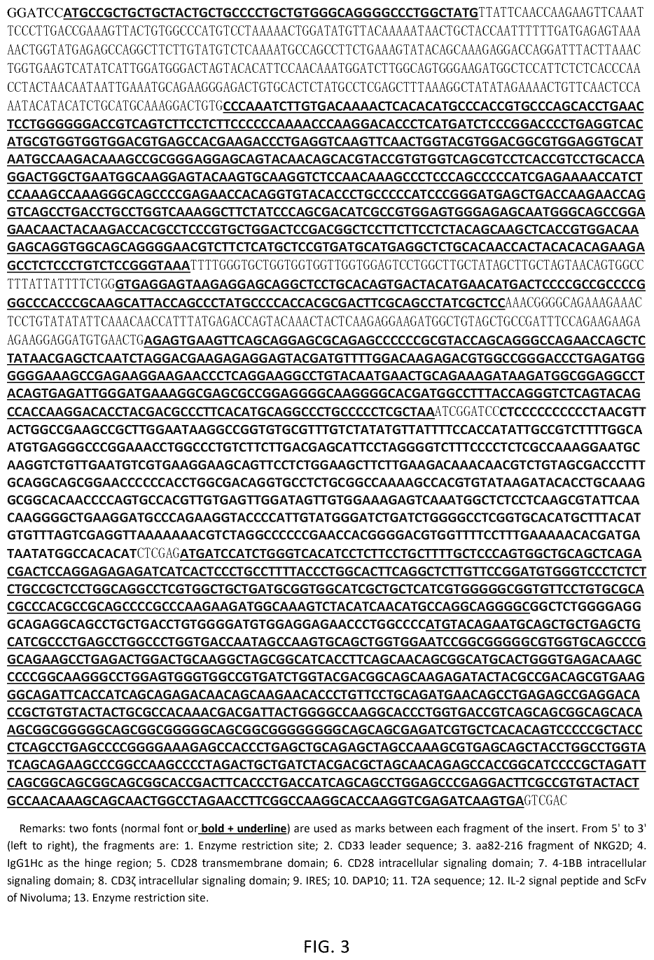

The construction methods and materials of pcD-NKG2D CAR-DAP10-anti-PD1-A are similar to pcD-NKG2D CAR-DAP10, including a nucleic acid fragment encoding NKG2D CAR and DAP10, as well as anti-PD1-ScFv as shown in FIG. 3. The nucleic acid fragment includes, from 5′ to 3′, the fragments: 1. CD3ζ leader sequence; 2. aa82-216 fragment of NKG2D; 3. IgG1Hc as the hinge region; 4. CD28 transmembrane domain; 5. CD28 intracellular signaling domain; 6. 4-1BB intracellular signaling domain; 7. CD3ζ intracellular signaling domain; 8. IRES; 9. DAP10; 10.T2A linker; 11. TL2 signal peptide; 12. Anti-PD1-ScFv fragment. The anti-PD1-ScFv fragment is a ScFv fragment derived from the Anti-PD1 antibody Nivolumab, which has the following amino acid sequence:

QVQLVESGGGVVQPGRSLRLDCKASGITFSNSGMHWVRQAPGKGLEWVAVIWY
DGSKRYYADSVKGRFTISRDNSKNTLFLQMNSLRAEDTAVYYCATNDDYWGQGT
LVTVSSGSTSGGGSGGGSGGGGSSEIVLTQSPATLSLSPGERATLSCRASQSVSSYL
AWYQQKPGQAPRLLIYDASNRATGIPARFSGSGSGTDFTLTISSLEPEDFAVYYCQ
QSSNWPRTFGQGTKVEIK.

There is a sequence encoding IL-2 signal peptide upstream of the nucleic acid encoding the above anti-PD1-ScFv fragment. The IL-2 signal peptide has the following amino acid sequence: MYRMQLLSCIALSLALVVTNS.

The above nucleic acid fragment was inserted downstream of the EF1α promoter of pCDH plasmid. The plasmid with the inserted fragment was transformed into competent E. coli, spreaded on a plate, and single clones were picked for sequencing and identification on the next day.

3. Construction of pcD-NKG2D CAR-DAP10-Anti-PD1-B

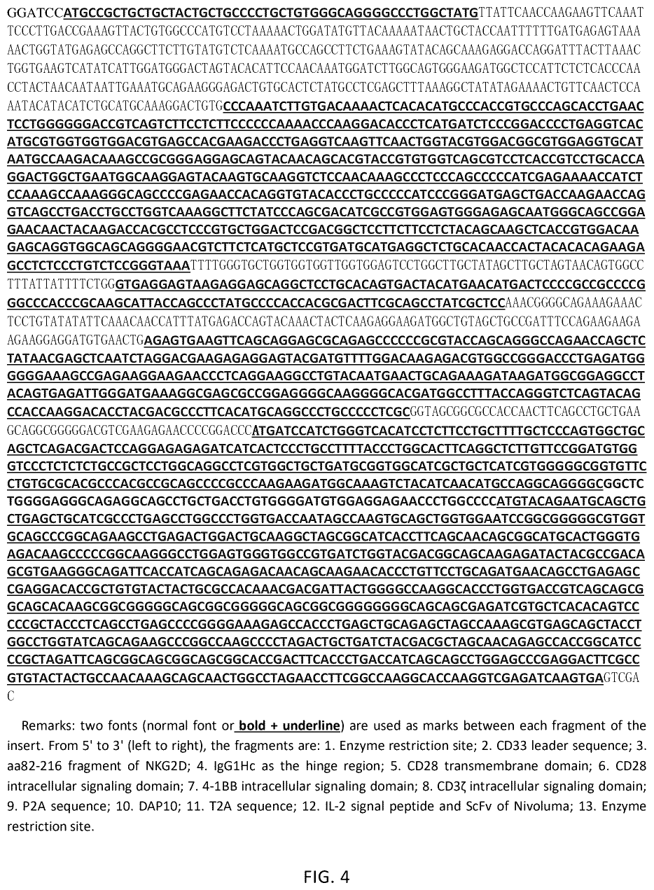

The construction methods and materials of pcD-NKG2D CAR-DAP10-anti-PD1-B are similar to pcD-NKG2D CAR-DAP10-anti-PD1-A, including a nucleic acid fragment encoding NKG2D CAR and DAP10, as well as anti-PD1-ScFv as shown in FIG. 4. The difference between the inserted fragments of pcD-NKG2D CAR-DAP10-anti-PD1-B and pcD-NKG2D CAR-DAP10-anti-PD1-A is that the linker between NKG2D CAR and DAP10 is through the P2A fragment instead of IRES. The nucleic acid fragment includes, from 5′ to 3′, the fragments: 1. CD33 leader sequence; 2. aa82-216 fragment of NKG2D; 3. IgG1Hc as the hinge region; 4. CD28 transmembrane domain; 5. CD28 intracellular signaling domain; 6. 4-1BB intracellular signaling domain; 7. CD3ζ intracellular signaling domain; 8. P2A; 9. DAP10; 10.T2A linker; 11. IL2 signal peptide; 12. Anti-PD1-ScFv fragment.

The above nucleic acid fragment was inserted downstream of the EF1α promoter of pCDH plasmid. The plasmid with the inserted fragment was transformed into competent E. coli, spreaded on a plate, and single clones were picked for sequencing and identification on the next day.

### Example 3 Construction of Lentiviral Vectors Expressing NKG2D CAR or Co-Expressing Anti-PD1

### 1. Production of Lentiviral Vectors

Using pcD-NKG2D CAR-DAP10, pcD-NKG2D CAR-DAP10-anti-PD1-A or pcD-NKG2D CAR-DAP10-anti-PD1-B as expression plasmids respectively, and third-generation lentiviral plasmids pMDLg/pRRE, pMD2.G and pRSV-Rev were co-transfected to prepare corresponding lentiviral vectors.

Using method recited in Example 1, expression of NKG2D-DAP10 and/or PD1-ScFv by the lentivirus were measured.

### Example 4: CAR-T Cells Co-Expressing NKG2D and Anti-PD1 Kill Cancer Cells In Vitro

According to the method described in Example 1, T cells were isolated from human PBMC. Then, the lentivirus containing pcD-NKG2D CAR-DAP10, pcD-NKG2D CAR-DAP10-anti-PD1-A or pcD-NKG2D CAR-DAP10-anti-PD1-B prepared in Example 3 was used to transduce T cells. The cytotoxicity assay was performed according to the method described in Example 1, and the specific cytotoxic effects of T cells expressing CAR on various tumor cells were examined. Therein, T cells prepared with NKG2D CAR-DAP10, NKG2D CAR-DAP10-anti-PD1-A or NKG2D CAR-DAP10-anti-PD1-B (referred to as NKG2D CAR T cells) and the T cells without transfected with lentivirus served as effector cells, while cancer cells (including myeloma NCI-H929 and NCI-H929, and lung cancer cell lines A549, NCI-H522 and NCI-H23) served as target cells.

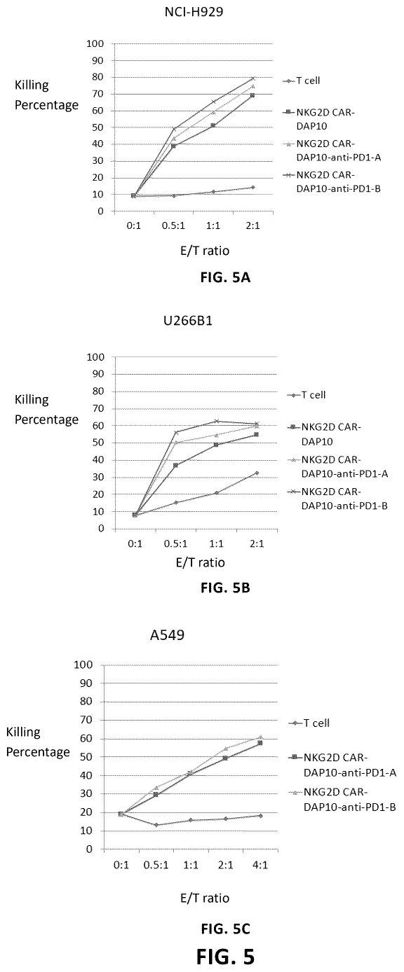

The results are shown in FIG. 5A-FIG. 5E. Under the same experimental conditions, compared with normal T cells that not comprising CAR, NKG2D CAR T cells significantly killed more NKG2D ligand expressing target tumor cells.

At the same time, the results also show that for a variety of tumor cells, CART cells expressing NKG2D CAR and DAP10 in combination with secretable anti-PD1-ScFv fragments can induce significantly more cell mortality on target tumor cells than CAR T cells expressing only NKG2D CAR and DAP10.

### Example 5 CART Cells Co-Expressing NKG2D and Anti-PD1 Co-Cultured with Target Cells Secrete IFN-γ

The CART cells prepared in Example 4 expressing NKG2D CAR-DAP10, NKG2D CAR-DAP10-anti-PD1-A or pcD-NKG2D CAR-DAP10-anti-PD1-B respectively and those not transfected with lentivirus T cells were collected and counted. Myeloma NCI-H929 and NCI-H929, and lung cancer cell lines A549, NCI-H522, and NCI-H23 were provided as targeting cancer cells. T cells or CAR-T cells were added to the 96-well plate, 100 μL per well, and then 0 μL, 25 μL, 50 μL, and 100 μL target cells were added respectively, and a control group was provided which only target cells were added. Supplement with the X-VIVO 15+5% HS+1% L-glutamine medium to 200 μL, and placed in a 37° C., 5% CO₂ incubator for 24 hours.

| T cell:target cell | 1:2 | 1:1 | 1:0.5 | 1:0 |
|---|---|---|---|---|
| target cell | 100 μL | 50 μL | 25 μL | 0 μL |
| T cell | 100 μL | 100 μL | 100 μL | 100 μL |

80 μL of supernatant was collected and the concentration of IFN-γ was detected according to the instructions of the Human IFN-γ ELISA set kit (BD).

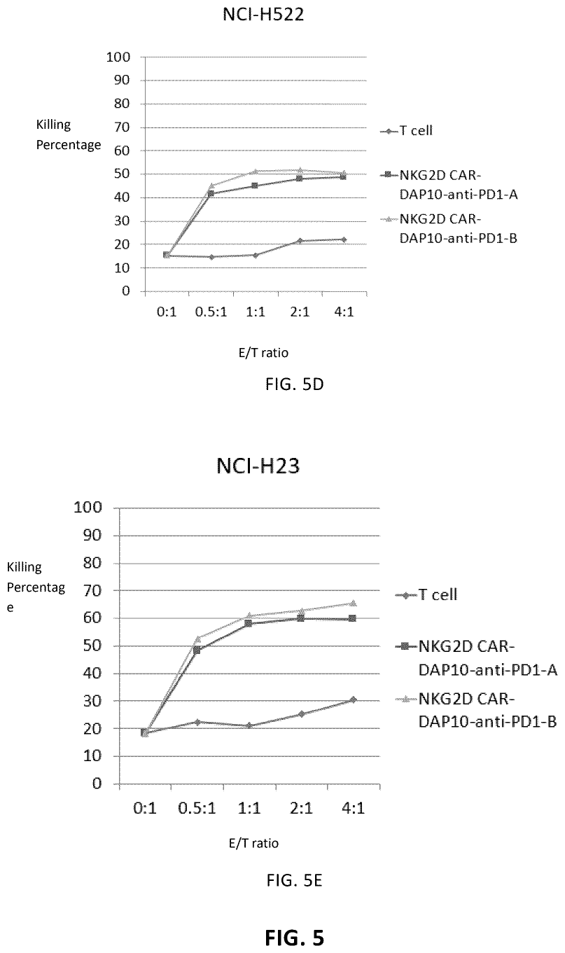

The results are shown in FIG. 6A to FIG. 6C. Under the same experimental conditions, compared with normal T cells without CAR, CAR T cells expressing NKG2D CAR and DAP10 and expressing secretable anti-PD1-ScFv fragments can significantly increase the generation of IFN-γ.

At the same time, the results also show that for a variety of tumor cells, CART cells expressing NKG2D CAR and DAP10 and secreted anti-PD1-ScFv fragments can increase the production of IFN-γ than CAR T cells expressing only NKG2D CAR and DAP10.

### Example 6: CART Cells Co-Expressing NKG2D and Anti-PD1 Inhibit Tumors in Human Lung Cancer Transplantation Animal Models

The CART cells respectively expressing NKG2D CAR-DAP10, NKG2D CAR-DAP10-anti-PD1-A and pcD-NKG2D CAR-DAP10-anti-PD1-B prepared in Example 4 were transplanted into human xenograft A549-luc model. In vivo pharmacodynamic studies were conducted.

This research was conducted by a third party, Yikang (Beijing) Pharmaceutical Technology Co., Ltd.

### Experimental Animals:

Species and strain: Mus Musculus, NCG. Age: 6-8 weeks; female; weight 18-22 g. Laboratory animal provider: Jiangsu Jicui Yaokang Biotechnology Co., Ltd.

Human lung cancer cells A549-luc labeled with luciferase were inoculated once into female NCG mice through the tail vein, and the cell inoculation volume was 1×10⁷/mouse. Three days after inoculation, the animals were randomly divided into groups according to the in vivo imaging signals with 5 animals in each group, a total of 5 groups, namely: Vehicle (iv, administered once) group, Vector-T (1×10⁷ cells/mouse, iv, once) group, CAR T cells expressing NKG2D CAR-DAP10 (CART, 1×10⁷ cells/mouse, iv, once) group, CAR T cells expressing NKG2D CAR-DAP10-anti-PD1-A (CART-L, 1×10⁷ cells/mouse, iv, once) group and CAR T cells expressing NKG2D CAR-DAP10-anti-PD1-B (CART-S, 1×10⁷ cells/mouse, iv, once) group.

### Detection Indicator:

1) Response of animals after administration: After grouping, weigh the mice twice a week, and record the relationship between the changes in the mice's weight and the administration time. At the same time, observe the survival and health status of the mice, such as animal activities during administration and other general conditions.
2) In vivo bioluminescence signal: After grouping, mice were intraperitoneally injected with luciferin substrate (15 mg/mL, 10 μL/g) once a week. After the mice were anesthetized with isoflurane, small animal in vivo imaging equipment (IVIS Lumina Series) was used. III, PerkinElmer) to collect luminescence signals and detect tumor growth characteristics in living animals, imaging a total of 6 times.
3) Survival period of tumor-bearing mice: observe the health status of mice, record the survival time of each mouse when it dies or reaches the euthanasia end point, and calculate the median survival time (MST) of tumor-bearing mice in each group and the median survival time (MST) of tumor-bearing mice in the treatment group The prolongation rate of survival (ILS %) is calculated as: (MST in the treatment group/MST-1 in the control group)×100%. The observation period ends 35 days after grouping.

### Biological Specimen Collection and Testing:

1) After the start of treatment, collect peripheral blood from mice once a week (from PG-D7) through the orbital venous plexus, namely PG-D7, PG-D14, PG-D21, PG-D28, and PG-D35, 3 mice each time A total of 58 animals were used to detect the proportion of human hCD3-positive cells (hCD3+%) by flow cytometry (FACS).
2) 32 days after the start of treatment (PG-D32) and at the end of the experiment (PG-D35), sera were collected from surviving animals when they were euthanized, a total of 4 copies, for subsequent ELISA detection of IFN-γ.
3) At the end of the experiment and when the experimental animals reach the euthanasia standard, for the surviving animals, gross dissection will be performed after euthanasia and the main organs of the mice (heart, liver, spleen, lung, kidney, brain) will be collected, a total of 10 parts, and HE stained. Observe pathological changes under the microscope; perform immunohistochemical staining (IHC) of CAR to observe whether there is any off-target.

The results are as follows.

1) Reactions and Body Weight Changes of Experimental Animals after Administration

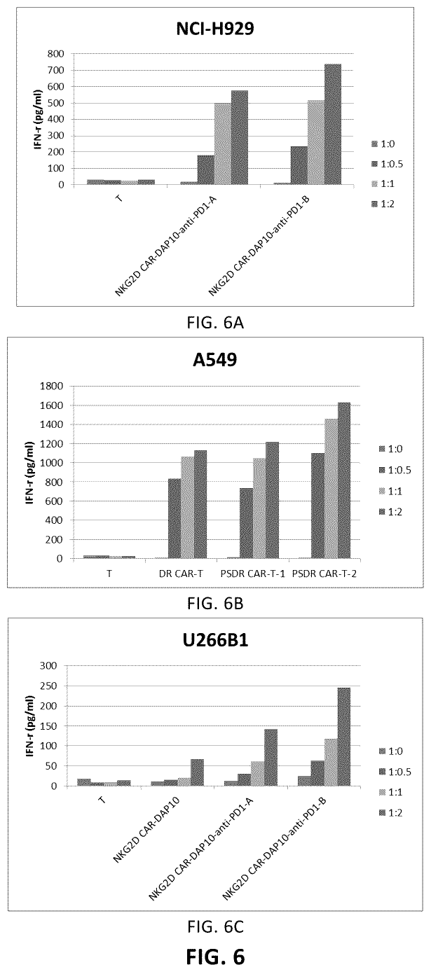

There were no obvious acute adverse reactions during administration of CART, CART-L, and CART-S. Mice in the Vehicle group and each treatment group gradually became ill and died starting from PG-D12 as the disease progressed. During the treatment period, the body weight of mice in the Vehicle group and Vector-T group showed a downward trend since 17 days after grouping (PG-D17); the body weight of mice in the CART group, CART-L group, and CART-S group was relatively stable during the experiment (FIG. 7).

### 2) In Vivo Imaging Observation of Mice

After tumor inoculation, the bioluminescence signal of mice in the Vehicle group gradually increased with the onset of tumors. The CART group, CART-L group, and CART-S group showed significant inhibition of tumor growth early after administration. By PG-D21 (i.e., 24 days after tumor inoculation), 2, 2, and 1 mice in the Vehicle group, Vector-T group, and CART-L group had died respectively, so the bioluminescence signal of PG-D14 was used for the experiment. Statistical analysis showed that the bioluminescence signal intensity of the CART group, CART-L group, and CART-S group was significantly lower than that of the Vehicle group, while there was no significant difference between the Vector-T group and the Vehicle group; there was no significant difference between treatment groups CART T-L group and CART-S group.

### 3) Survival Period of Tumor-Bearing Mice

The experiment ended at 38 days after cell inoculation (PG-D35) for survival observation. The mice in Vehicle group and the Vector-T group died successively since PG-D12 and PG-D19 respectively, with a median survival time of 24 days. The mice in the CART group, CART-L group, and CART-S group became ill and died successively from PG-D28, PG-D14, and PG-D28 respectively. By the end of the survival period observation at PG-D35, one experimental animal in each group survived. The median survival times of the Vector-T group, CART group, CART-L group and CART-S group were 24 days, 30 days, 31 and 32 days respectively, and the survival extension rates were 0%, 25%, 29% and 33% respectively.

### Example 7 CART Cells Co-Expressing NKG2D and Anti-PD1 Inhibit Tumors in Clinical Trials

The NKG2D CAR-DAP10 and NKG2D expressed respectively prepared in Example 4 were CAR-DAP10-anti-PD1-A and pcD-NKG2D CAR-DAP10-anti-PD1-B CAR T cells were subjucted to clinical testing.

The clinical study was approved by the Ethics Review Committee of Peking University First Hospital and registered with the China Clinical Trial Registration Center. The subjects signed a formal informed consent form, and non-small cell lung cancer (NSCLC) patients and normal controls were recruited according to the inclusion and exclusion criteria. The subjects and normal controls were randomly assigned to the test group according to the random number comparison table. and control group.

The above disclosures show that the CAR and CAR-T cells with NKG2D antigen receptor structure provided by the present invention can effectively recognize cancer cells with NKG2D ligands and activate tumor cell-specific anti-tumor cell immune responses and killing-related tumor cells. Experiments also prove that the CAR and CAR-T cells with the NKG2D antigen receptor structure provided by the present invention, when combined with the secretable anti-PD-1 scFv, can significantly enhance the effect of killing tumors, including solid tumors (including lung cancer and myeloma, etc.).

Unless otherwise indicated, the practice of the present disclosure will employ common technologies of organic chemistry, polymer chemistry, biotechnology, and the like. It is apparently that in addition to the above description and examples than as specifically described, the present disclosure can also be achieved in other ways. Other aspects within the scope of the disclosure and improvement of the present disclosure will be apparent to the ordinary skilled in the art. According to the teachings of the present disclosure, many modifications and variations are possible, and therefore it is within the scope of the present disclosure.

Unless otherwise indicated herein, the temperature unit “degrees” refers to Celsius degrees, namely ° C.

1.-17. (canceled)

18. An immune cell that expresses,
i) a chimeric antigen receptor (CAR),
ii) a PD-1 antibody or an active fragment thereof, and
iii) a DAP10 or active fragment thereof,
wherein the CAR comprises: (a) an antigen binding domain comprising an NKG2D or a fragment thereof;
(b) a transmembrane domain; and (c) an intracellular signaling domain.

19. The immune cell of claim 18, wherein ii) is a single chain variable fragment (scFv), Fab, F(ab′)2, Fv, Fd or dAb of a PD-1 antibody.

20. The immune cell of claim 19, wherein the PD-1 antibody or active fragment thereof is secretable.

21. The immune cell of claim 18, wherein said transmembrane domain is a transmembrane domain of CD8 and/or CD28.

22. The immune cell of claim 18, wherein said intracellular signaling domain is an intracellular signaling domain comprising CD28, 4-1BB and CD3ζ.

23. The immune cell of claim 18, further comprising a hinge region between said antigen binding domain and said transmembrane domain, which is an IgG heavy chain constant region fragment (IgGHc).

24. The immune cell of claim 18, wherein the antigen binding domain comprising an NKG2D fragment corresponding to amino acid residues 82-216.

25. The immune cell of claim 18, wherein a leading peptide is located upstream of the NKD2G or a fragment thereof.

26. The immune cell of claim 18, wherein the immune cell is a T cell, a natural killer T (NKT) cell, a natural killer (NK) cell, a human embryonic stem cell, a hematopoietic stem cell (HSC), or an induced pluripotent stem cell (iPS).

27. The immune cell of claim 26, wherein the immune cell is autologous.

28. The immune cell of claim 18, which is for the use of treating or preventing cancer.

29. The immune cell of claim 28, wherein the cancer is leukemia, lymphoma, or solid tumor.

30. The immune cell of claim 28, wherein the cancer is myeloma or lung cancer.

31. An isolated nucleic acid comprising a nucleotide sequence encoding the following components expressed by a cell as defined in claim 18:
i) a chimeric Antigen Receptor (CAR),
ii) a PD-1 antibody or an active fragment thereof, and
iii) DAP10 or active fragment thereof,
wherein the CAR comprises: (a) an antigen binding domain comprising an NKG2D or a fragment thereof;
(b) a transmembrane domain; and (c) an intracellular signaling domain.

32. The nucleic acid of claim 31, wherein the an antigen binding domain comprising an NKG2D fragment corresponding to amino acid residues 82-216.

33. The nucleic acid of claim 31, wherein ii) is a single chain variable fragment (scFv), Fab, F(ab′)2, Fv, Fd or dAb of a PD-1 antibody.

34. The nucleic acid of claim 31, wherein there is an IRES or 2A sequence between the nucleic acid encoding the CAR and/or DAP10 and the nucleic acid encoding the PD-1 antibody or an active fragment thereof.

35. The nucleic acid of claim 31, which is for the use of treating or preventing cancer.

36. The nucleic acid of claim 35, wherein the cancer is leukemia, lymphoma, or solid tumor.

37. The nucleic acid of claim 35, wherein the cancer is myeloma or lung cancer.
